(19) United States
(12) Patent Application Publication
(10) Pub. No.: US 2006/0116657 A1
(43) Pub. Date: Jun. 1, 2006

(54) Mixing capsule

(76) Inventor: Daniel Schmid, Gossau (CH)

Correspondence Address: NIXON & VANDERHYE, PC, 901 NORTH GLEBE ROAD, 11TH FLOOR, ARLINGTON, VA, 22203, US

(21) Appl. No.: 10/550,200

(22) PCT Filed: Mar. 19, 2004
(86) PCT No.: PCT/CH04/00171

§ 371 Date: Dec. 29, 2005

(30) Foreign Application Priority Data
Mar. 21, 2003 (CH) 472/03

(51) Int. Cl. A61B 19/00

(52) U.S. Cl. 604/416

(57) ABSTRACT

The invention relates to a mixing capsule (11), for a two-component mixture, comprising a container piece (13) and an ejection nozzle (19) on the front face thereof. A piston (15) may be axially displaced in the container piece (13). A through opening (55) is provided in the front face of the piston (15), to which a first chamber (53) is connected. The cavity between the front face of the container piece (13) and the front face of the piston (15) defines a second chamber or a mixing chamber (35). In the unused state of the capsule (11), a bursting membrane (59) seals the through opening (55) between the first and the second chamber. The mixing capsule (11) and the ejection nozzle (19) are embodied in at least two pieces and connected to each other by means of detachable connector means.

With regard to point V

Reasoned findings with regard to novelty, inventive step and industrial applicability; documents and arguments to support these findings
1. The following documents are referred to: D1: EP 1219262 A D2: WO 01/62317 A
2. The present application does not fulfil the requirements of Article 33(1) PCT, since the subject-matter of claim 1 is not new within the meaning of Article 33(2) PCT
   1. Document D1 discloses (the references in brackets relate to this document):

A mixing capsule (1) for accommodating a two-component mixture, for manufacturing a dental mass, with a cylindrical container part (1) with an end-face (1d) with an ejection nozzle (4), and with an open rear side (see figures) lying opposite to the end-face, having an opening (see Figures) for inserting a movable piston (2), wherein a first chamber (1a) is defined between the end-face (1d) of the container part (1) and the piston (2), said first chamber serving for accommodating a first, preferably powder-like component (A) of a multi-component mixture; with a second chamber (2a) provided on or in the piston (2), for accommodating a second, preferably fluid or at least flowable component (B) of a multi-component mixture, with a through-opening (see figures) between the first and the second chamber (1a, 1b) which is closed in the initial condition by way of a destructible wall (2e), as well as with a means (3) in order to open the initially closed through-opening between the first and the second chamber (1a, 1b) and to transfer the contents (B) of the one receptacle into the other chamber, wherein the mixing capsule (1) and the ejection nozzle (4) are deigned at least of two pieces and are connectable to one another by way of detachable fastening means (see figures)

2. The dependent claims 2-13 contain no features which in combination with any other claim to which they relate, fulfil the requirements of the PCT with regard to novelty and inventive step, see documents D1 and D2 and the corresponding text locations cited in the search report.

The invention relates to a mixing capsule for accommodating a two-component mixture, in particular for manufacturing a dental mass, according to the preamble of claim 1.

A multitude of mixing capsules for curable dental masses is known in the state of the art. Examples of mixing capsules are for example disclosed in the following patent applications: EP-A-0 245 788, U.S. Pat. No. 5,026,283, DE 43 115 920 and DE-A-39 20 537.

The mentioned mixing capsules serve for the storage and mixing of the initial components of a curable multi-component mixture. As soon as the initial components are mixed with one another, a spontaneous polymerisation reaction sets in, and a hard material which serves as a dental filling forms from the initial components within minutes.

In dental technology today, one uses a multitude of different multi-component mixtures depending on the purpose of application. These multi-component mixtures are viscous or flowable to a differing extent, so that the dimensioning of the mixing capsules must be matched in each case to the viscosity of the multi-component mixture. In particular, the ejection nozzle needs to be dimensioned such that the multi-component mixture may be pressed out of the mixing capsule with a small force effort.

The multi-component mixture, which is manufactured in the mixing capsule directly before the actual application, is pressed out by the dentist into the cavity of a tooth to be repaired, with a known pressing-out pistol. With this, the operating dentist must allow the ejection nozzle of the mixing capsule to project as far as possible into the cavity of the tooth, so that the cavity is completely filled on pressing out the mass. This however, with the treatment of the rear molars, is often very difficult to accomplish on account of the size of the pressing-out pistol and the stiffness as well as limited length of the ejection nozzle.

Since much skilful handwork is required for the profession of the dentist, it is obvious that a dentist values tools which lie comfortably in the hand and also permit an optimal working even under difficult conditions. For this reason too, the most varied of pressing-out pistons have been provided in the past for pressing out the multi-component mixtures prepared in the mixing capsules, which are to simplify the application of multi-component mixtures. With regard to the mixing capsules themselves, although those with differently designed ejection nozzles are known, these however in each case are firmly connected to the mixing capsule.

A common feature of the known mixing capsules is the fact that the ejection nozzles are in each case of one piece with the mixing capsule.

Syringes with exchangeable tips have been known for many years. The syringes serve for storing the most varied of solutions, such an etching fluids.

It is therefore the object of the present invention to provide a mixing capsule which may be applied for processing multi-component mixtures of a different viscosity. A further aim is to provide a capsule which permits an application of the multi-component mixture also at difficultly accessible locations. It is yet an object to suggest a capsule which may be matched to the needs of the respective required treatment on location.

According to the invention, a mixing capsule is characterised in that the mixing capsule and the ejection nozzle are formed of at least two pieces and may be connected to one another by way of releasable fastening means. The mixing capsule according to the invention has the advantage that one may use different ejection nozzles depending on the viscosity of the flowable two- or multi-component mixture manufactured in the capsule. The manufacturer of the multi-component mixtures is therefore no longer forced to apply different mixing capsules depending on the viscosity of the mixture, but one may always use one and the same mixing capsule—but with a different ejection nozzle. One may thus leave it to the dentist to attach ejection nozzles designed differently long, on the mixing capsule, depending on the location of application.

Advantageously, the ejection nozzle on the mixing capsule side comprises a flange which is sealingly connectable to the mixing capsule. By way of this one may prevent material from unintentionally exiting at the connection location. Basically, the ejection nozzle may be capable of being stuck onto the mixing capsule with the flange (push-on connection). This permits a rapid attachment or exchange of the ejection nozzle.

According to a particularly preferred embodiment, a connection stub with a helical inner thread is provided on the mixing capsule. A connection part with a corresponding outer thread may also be provided on the ejection nozzle. The ejection nozzle may be reliably fixed with a screw connection. A screw connection has the advantage over a snap or push-on connection that the ejection nozzle fastened in such a manner may not be unintentionally detached. If specifically the ejection nozzle were not to bear tightly on the mixing capsule, then although material may exit, this then is without the ejection nozzle detaching itself.

According to a particularly preferred embodiment, a double helical thread with the same pitch is provided on the stub, whose paths begin offset by 180 degrees to one another. This has the advantage that with a firmly screwed nozzle, a uniform pressing pressure is present on oppositely lying sides of the flange. The flange is advantageously oval, and may cooperate with the double helical inner thread of the connection stub.

Usefully, a stub is integrally formed at the exit opening of the mixing capsule, and the ejection nozzle has a widened connection part which may be capable of being placed onto the stub. The ejection nozzle may be pushed onto the mixing capsule in an ideal manner by way of this guiding possibility, in particular if the stub projects beyond the connection stub by a certain measure.

The exchangeable ejection nozzle described above may be provided with mixing capsules designed in the most varied of types. These mixing capsules are conceived for accommodating dental masses, and in the initial condition in each case comprise two chambers which are separated from one another, in which the initial components of a two or multi-component mixture which may be polymerised spontaneously or initiated by light are stored. Such mixing capsules as a rule have an axially displaceable activation element, with which the wall closing the through-opening may be destroyed or opened.

The invention is hereinafter described by way of example with the reference to the figures. There are shown in:

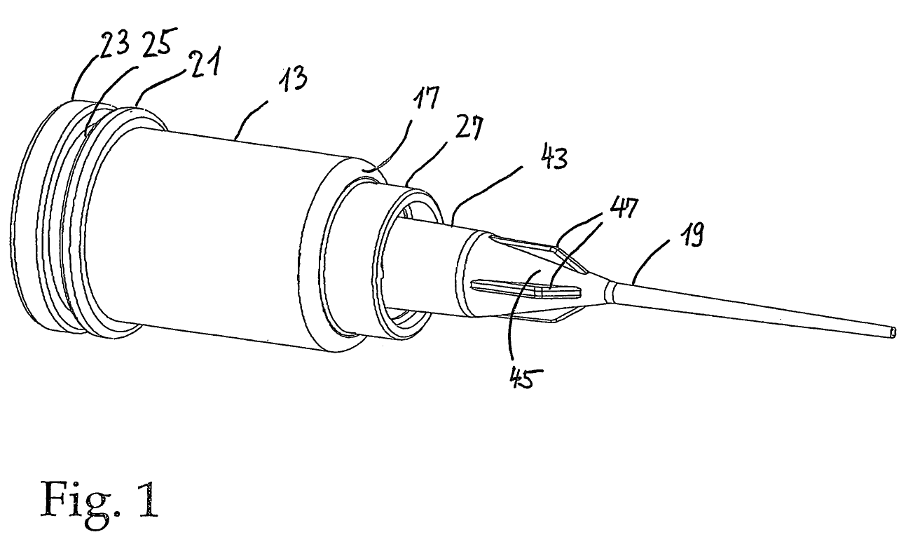

FIG. 1: a mixing capsule according to the invention with an exchangeable ejection nozzle, in a perspective view;

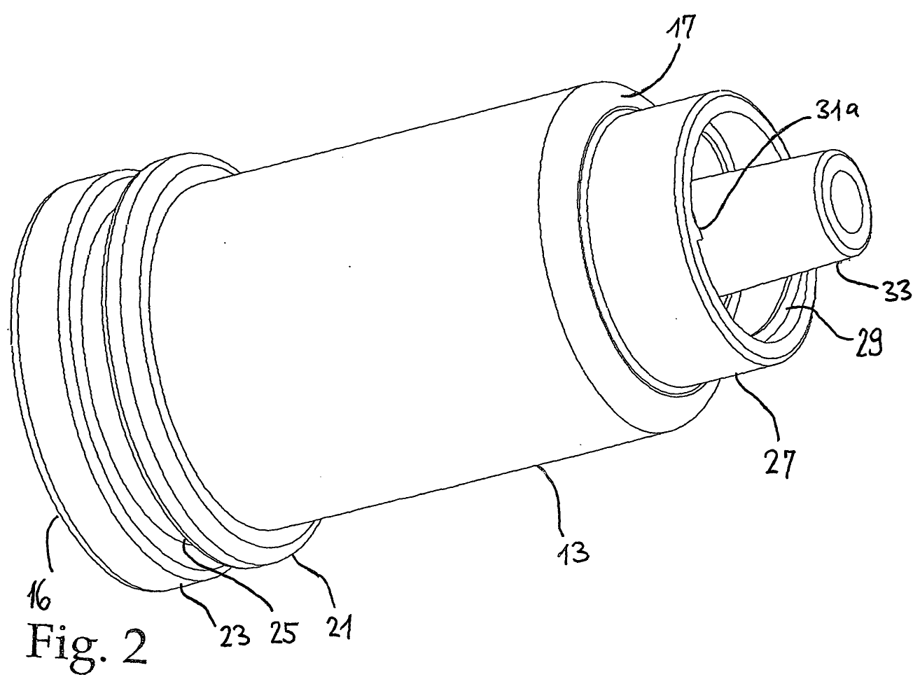

FIG. 2: the mixing capsule of FIG. 1, without an ejection nozzle:

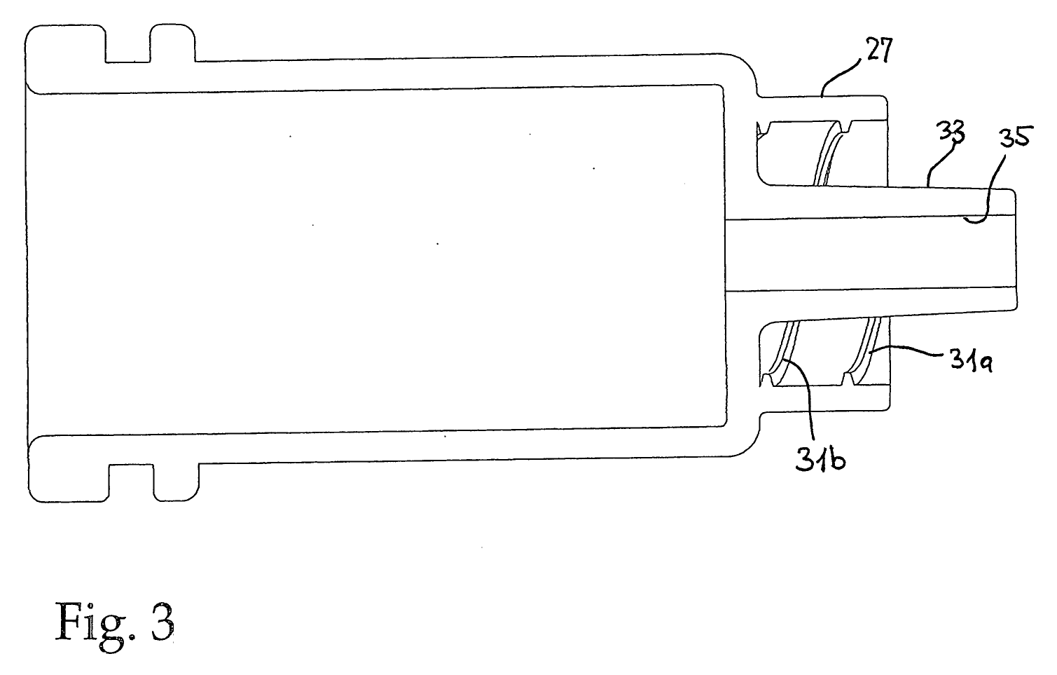

FIG. 3: the mixing capsule of FIG. 2 in a longitudinal section;

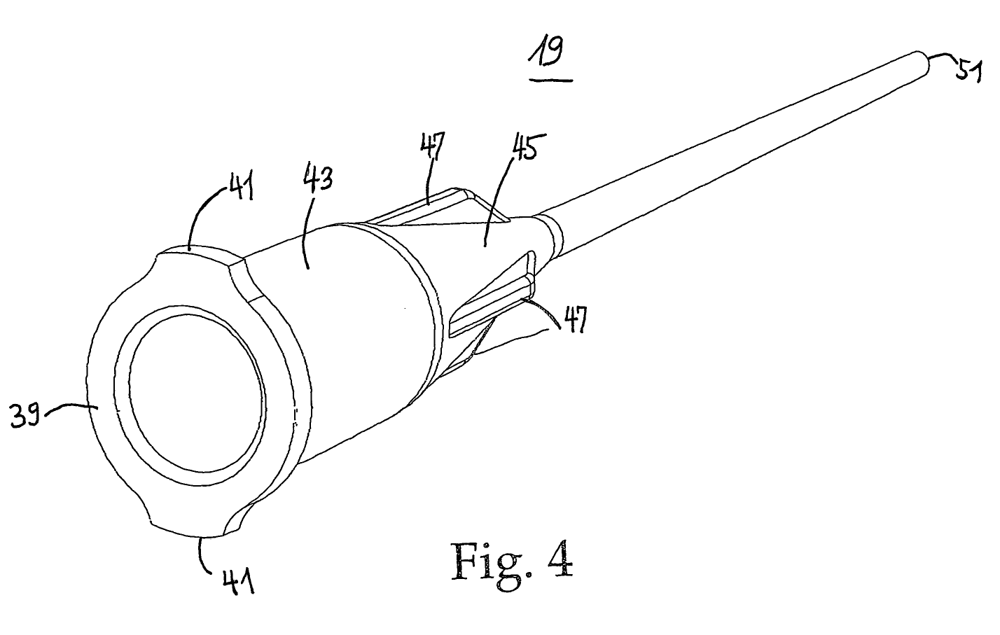

FIG. 4: the ejection nozzle of FIG. 1 in a perspective view;

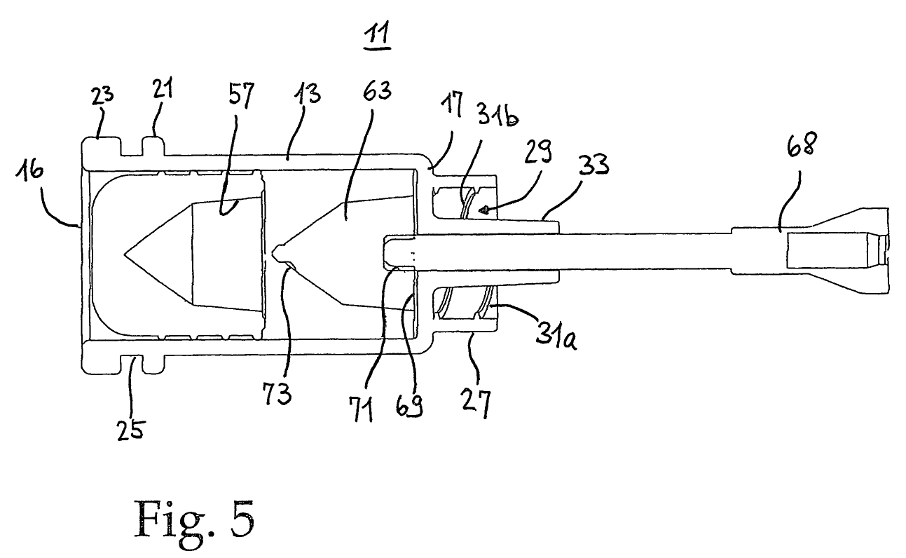

FIG. 5: a mixing capsule according to the invention without an ejection nozzle, but with activation means in the initial position;

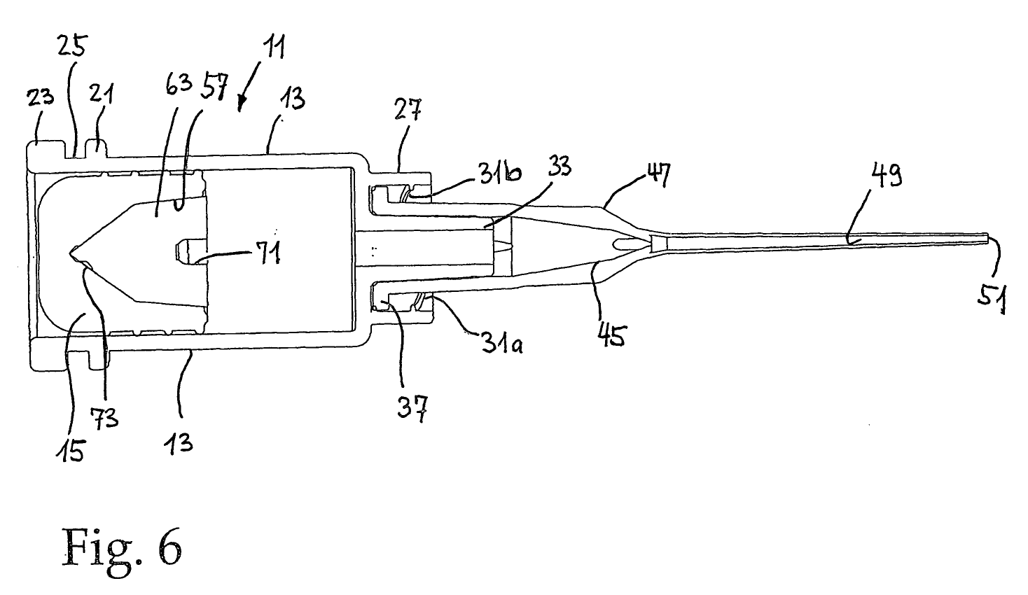

FIG. 6: the mixing capsule of FIG. 5, with the activation means in the activation position.

FIGS. 1 to 6 show a mixing capsule 11 according to the invention, with an external container part 12 and a piston 15 accommodated in the container part 13. The piston 15 is axially displaceable in the container part 13 and in FIG. 5 is located in the initial position or filled position, and in FIG. 6 in the mixing position (activated condition of the capsule). The container part 13 is cylindrical and has an opening 16 for introducing the piston 15, and an end-face 17 on which an ejection nozzle 19 is detachably (releasably) arranged. An annular groove 25 is present at the rear end of the container part between two annular shoulders 21, 23 and this groove serves for receiving one jaw of a pressing-out tool known in the field.

According to the invention, the ejection nozzle 19 is not formed as one piece with the mixing capsule container 12, but as an individual part which is detachably fastenable on the end-face 17 of the container 13. Connection means, preferably in the form of a rotational or a screw connection, are provided for connecting the ejection nozzle 19 to the container 13. According to the shown preferred embodiment example, a connection stub 27 with a helical inner thread 29 is integrally formed on the container 13. The inner thread 29 is designed as a double (two part) thread with two screw paths 31a, 31b (FIG. 3) offset by 180 degrees to one another. Basically one could also use a three- or multi-part thread with screw paths which are offset to one another by 120 degrees or accordingly less. A stub 33 is provided in the centre of the connection stub 27. An exit channel 35 is formed in the stub 33 and this channel runs into the mixing capsule 11. The stub 33 projects beyond the connection stub 27 to the front by a certain amount. This simplifies the connection of the ejection nozzle 19 to the mixing capsule 11.

The ejection nozzle 19 at the connection side has a connection flange 27 with a flat, annular sealing surface 29. Two ears 41 are integrally formed on sides of the connection flange 37 which lie opposite one another. These ears may be engaged by the screw paths 31a, 31b. The connection flange 37 according has an essentially oval shape. By way of the screw paths 31a, 31b which are offset to one another by 180 degrees, the ears 41 and thus the sealing surface 39 of the connection flange 37 are pressed at oppositely lying sides uniformly onto the end-face 17 of the container 18 when the ejection nozzle 19 is screwed on. By way of this, it is ensured that on pressing a viscous or pasty mass out of the mixing capsule, no material may exit at the connection location between the ejection nozzle 19 and the container 13. Basically it is conceivable to apply a simple rotational closure without a screw thread in place of a screw connection.

The ejection nozzle 19 has a cylindrically formed connection part 43 whose inner diameter corresponds roughly to the outer diameter of the stub 33. Several longitudinal ribs 47 are formed at the outside on the connection part 43. These have the purpose of ensuring a good gripping on screwing the ejection nozzle onto the mixing capsule. The connection part 43 on the inside comprises a conical nozzle channel section 43 which runs into a nozzle channel 49. The ejection nozzle 19 has an exit opening 51 at the front.

Although the nozzle 19 may be connected to any mixing capsules, here for the sake of completeness, one mixing capsule is described by way of example, which is to represent all other types. As is particularly evident from the FIGS. 5 and 6, a first chamber 52, hereinafter called mixing chamber, is defined in the mixing capsule 11 between the piston 15 and the end-face 17, and this serves the accommodation of a preferably powder-like component of a multi-component mixture. Furthermore, a second chamber 53 is formed in the piston 15, and serves for accommodating a fluid or at least flowable component of a multi-component mixture. The second chamber 53 has an inner space 57 with a through-opening 55 which is directed to the end-face 17. In the non-activated condition according to FIG. 5, the through-opening 55 is closed with a film or membrane 59. The film may be welded onto the end-face 61 of the piston 15 in the known manner. One or more annular seals 62 are integrally formed on the piston casing for sealing the piston 15 towards the container part 13. The first seal 62 is located at the frontmost piston edge. Two further seals 62a, 62b are located at a distance to the first seal 62.

A displacement body 63 is applied into the first chamber 52 between the end-face 61 of the piston 15 and the end-face 17 of the container part 13. The displacement body 63 has a shape which is complimentary to the inner space 57 of the piston 15. The displacement body 63 is axially displaceable (see FIG. 5) in the container part 13 by way of an activation pin 67 which is accommodated in the ejection nozzle 19 before the activation of the mixing capsule. The length of the activation pin 67 is at least so long that the displacement body 63 may be completely pushed into the second chamber 53. The activation pin 67 has a head 68 which serves as an abutment.

The displacement body 63 on the base 69 has a round recess 71. The recess 71 serves for receiving the front part of the activation pin 67. Advantageously, the front part of the activation pin 67 and the recess 71 are created such that a friction fit is realised. By way of this, the displacement body 63 is fixed in the mixing space of the non-activated capsule 11. In order to ensure an unhindered flow of the fluid present in the second chamber into the mixing space 35, an overflow channel 73 (FIGS. 5 and 6) is provided in the casing of the displacement body.

The exchangeable ejection nozzle 19 may just as easily be provided on a mixing capsule as is described in the initially mentioned patent applications. These mixing capsules likewise have two chambers for the accommodation of the initial components of a polymerisable multi-component mixture. As described in DE 43 15 920, a piston axially displaceable in a mixing capsule may be designed as a hollow piston. In turn, a plunger (punch) may be arranged in this hollow piston in an axially displaceable manner. The axially displaceable plunger serves as an activation part which on advance may destroy a burstable fluid receptacle arranged in the hollow piston, so that the contents of the receptacle pour into the mixing space. The mixture present in the mixing space may be pressed through the ejection nozzle by way of a subsequent displacement of the piston 15.

A mixing capsule for a two-component mixture has a preferably cylindrical container part 13 with at least one (first) piston 15 which is axially displaceable in the container part 13. The mixing capsule 11 in the initial condition has two chambers which serve for the accommodation of two initial components of a multi-component mixture which—when brought together—may be spontaneously polymerised. An exchangeable ejection nozzle 19 is provided at the end-face of the mixing capsule 11. The ejection nozzle 19 is detachably connected to the mixing capsule. This has the advantage that a suitable application nozzle may be assembled, depending on the viscosity of the mixture and the conditions of application. A second piston (not shown in the figures) may then be arranged in the piston 15 in an axially displaceable manner. A second chamber is defined between the end-face of the second piston and the end-face of the first piston 15, and this second chamber serves for accommodating a flowable or fluid mass. With this, the mass stored in the second chamber may be displaced into the first chamber with the second piston.

### LIST OF REFERENCE NUMERALS

11 mixing capsule
13 container part
15 piston
16 opening in the container part 13 for introducing the piston
17 end-face of the container part
19 ejection nozzle
21, 23 annular shoulders
25 annular groove
27 connection stub
29 inner thread
31a, 31b screw paths
33 stub
35 exit channel
37 connection flange
39 sealing surface
41 ears
43 cylindrically formed connection part of the ejection nozzle
45 conical nozzle channel section
47 ribs
49 nozzle channel
51 exit opening
52 first chamber (mixing space)
53 second chamber
55 through-opening
57 inner space
59 membrane
61 end-face of the piston
62 ring seals
63 displacement body
67 activation pin
69 head of activation pin
71 recess
73 overflow channel

1. A mixing capsule (11) for receiving a two-component mixture, in particular for the manufacture of a dental mass with
a preferably cylindrical container part (13) with an end-face (17) with an ejection nozzle (19), and with an open rear side lying opposite the end-face (17), having an opening (16) for inserting a movable piston (15)
at least one piston (15) axially movable in the container part (13), wherein a first chamber (52) is defined between the end-face of the container part (13) and the piston (15), said first chamber serving the accommodation of a first, preferably powder-like component of a multi-component mixture;
a second chamber (53) provided on or in the piston (15) for accommodating a second, preferably fluid or at least flowable component of the mentioned multi-component mixture,
a through-opening (55) between the first and the second chamber (52, 53), which in the initial condition is closed by a destructible wall (59) as well as
a means (67) in order to open the initially closed through-opening between the first and the second chamber and to transfer the contents of the one receptacle into the other chamber,
further characterised in that
the mixing capsule and the ejection nozzle (19) are designed at least of two pieces and are connectable to one another by way of a releasable fastening means (29, 31, 37).

2. A mixing capsule according to claim 1, characterised in that the ejection nozzle (19) on the mixing capsule side comprises a flange (37) which may be sealingly connected to the mixing capsule (11).

3. A mixing capsule according to claim 1 or 2, characterised in that the ejection nozzle (19) is capable of being stuck onto the mixing capsule (11) with a flange (37).

4. A mixing capsule according to one of the claims 1 to 3, characterised in that a connection stub (27) with a helical inner thread (29) is provided on the mixing capsule (11).

5. A mixing capsule according to claim 4, characterised in that at least one double helical thread (31a, 31b) is provided on the connection stub (27), whose paths begin offset by 180 degrees to one another.

6. A mixing capsule according to claim 5, characterised in that the flange (37) is oval and capable to cooperate with the double helical inner thread (31a, 31b) of the connection stub (27).

7. A mixing capsule according to one of the claims 1 to 6, characterised in that a stub (33) is integrally formed on the end-face (17) of the mixing capsule (11), and that the ejection nozzle (19) has a widened connection part (43) which is capable of being placed onto the stub (33).

8. A mixing capsule according to claim 7, characterised in that the stub (33) projects beyond the connection stub (27) by a certain amount.

9. A mixing capsule according to one of the claims 1 to 8, characterised in that the means for opening the through-opening between the first and the second receptacle is formed by an axially displaceable activation element (67).

10. A mixing capsule according to one of the claims 1 to 9, characterised in that the through-opening (55) is closed with a membrane (59).

11. A mixing capsule according to claim 9 or 10, characterised in that the activation element is an axially displaceable activation pin (67) or an axially displaceable plunger.

12. A mixing capsule according to one of the claims 1 to 9, characterised in that the ejection nozzle (19) has a connection part (43) with a conical nozzle channel section (45), said nozzle channel section (45) running into a nozzle channel (49).

13. The use of a mixing capsule (11) according to one of the claims 1 to 12 for receiving the initial components of a curable, in particular spontaneously polymerisable dental mass.
